SEQUENCE LISTING (3 sequences)

SEQ ID NO: 1 - DNA, 39 bp, Artificial
  Template
  misc_feature (1)..(2): n is a, c, g, or t
  misc_feature (4)..(5): n is a, c, g, or t
  misc_feature (7)..(8): n is a, c, g, or t
  misc_feature (10)..(11): n is a, c, g, or t
  misc_feature (13)..(14): n is a, c, g, or t
  misc_feature (19)..(20): n is a, c, g, or t
  misc_feature (22)..(23): n is a, c, g, or t
  misc_feature (25)..(26): n is a, c, g, or t
  misc_feature (28)..(29): n is a, c, g, or t
  misc_feature (34)..(35): n is a, c, g, or t
  misc_feature (37)..(38): n is a, c, g, or t
nnknnknnknnknnksctnnknnknnknnkgtgnnknnk

SEQ ID NO: 2 - DNA, 21 bp, Artificial
  Template
  misc_feature (1)..(2): n is a, c, g, or t
  misc_feature (4)..(5): n is a, c, g, or t
  misc_feature (7)..(8): n is a, c, g, or t
  misc_feature (10)..(11): n is a, c, g, or t
  misc_feature (13)..(14): n is a, c, g, or t
  misc_feature (16)..(17): n is a, c, g, or t
  misc_feature (19)..(20): n is a, c, g, or t
nnknnknnknnknnknnknnk

SEQ ID NO: 3 - DNA, 82 bp, Artificial
  Template
  misc_feature (35)..(36): n is a, c, g, or t
  misc_feature (38)..(39): n is a, c, g, or t
  misc_feature (41)..(42): n is a, c, g, or t
  misc_feature (44)..(45): n is a, c, g, or t
  misc_feature (47)..(48): n is a, c, g, or t
  misc_feature (50)..(51): n is a, c, g, or t
  misc_feature (53)..(54): n is a, c, g, or t
aactttgtggaccacaccagctccatctcctaacnnknnknnknnknnknnknnkgatgctaaactcactttggtcttaaca

(19) United States
(12) Patent Application Publication
(10) Pub. No.: US 2022/0251548 A1
(43) Pub. Date: Aug. 11, 2022

(54) METHODS FOR PRODUCING A LIBRARY OF BIOLOGICAL MOLECULES

(71) Applicant: REGENTS OF THE UNIVERSITY OF MINNESOTA, Minneapolis, MN (US)

(72) Inventors: Praveensingh Bheemsingh Hajeri, Falcon Heights, MN (US); Masato Yamamoto, Golden Valley, MN (US)

(21) Appl. No.: 17/616,521

(22) PCT Filed: Jun. 5, 2020
(86) PCT No.: PCT/US2020/036300

§ 371 Date: Dec. 3, 2021

Related U.S. Application Data
(60) Provisional application No. 62/857,993, filed on Jun. 6, 2019.

(51) Int. Cl. C12N 15/10; C12N 1/20; C12N 15/70; C12P 19/34

(52) U.S. Cl. CPC C12N 15/1082; C12N 15/1093; C12N 1/20; C12Q 1/6869; C12P 19/34; C12N 2710/10343; C12N 2800/101; C12N 15/70

(57) ABSTRACT

A method for producing a library of biological molecules generally includes providing an engineered polynucleotide vector, amplifying the polynucleotide, and sequencing the library of biological molecules produced. The polynucleotide includes a cloning vector backbone, a recombination site, one or more cloning sites, and a nucleotide sequence template. The nucleotide sequence template includes a coding region, a sequence 5′ to the coding region that is complementary to a portion of the vector backbone, and a sequence 3′ to the coding region that is complementary to a portion of the vector backbone.

### CROSS-REFERENCE TO RELATED APPLICATION

This application claims the benefit of U.S. Provisional Patent Application No. 62/857,993, filed Jun. 6, 2019, which is incorporated herein by reference in its entirety.

### SUMMARY

This disclosure describes, in one aspect, a method for producing a library of biological molecules. Generally, the method includes providing an engineered polynucleotide vector, amplifying the polynucleotide, and sequencing the library of biological molecules produced. The polynucleotide includes a cloning vector backbone, a recombination site, one or more cloning sites, and a nucleotide sequence template. The nucleotide sequence template includes a coding region, a sequence 5′ to the coding region that is complementary to a portion of the vector backbone, and a sequence 3′ to the coding region that is complementary to a portion of the vector backbone.

In some embodiments, the nucleotide sequence of the coding region is the biological molecule on which the library is based. In other embodiments, the nucleotide sequence of the coding region encodes the biological molecule on which the library is based.

In some embodiments, amplifying the polynucleotide includes introducing the polynucleotide into a host cell, incubating the genetically modified cell under conditions to allow the polynucleotide to replicate, collecting genetically modified cells, and isolating vector from the collected cells. In other embodiments, amplifying the polynucleotide includes amplifying the polynucleotide in vitro.

In some embodiments, the nucleotide sequence includes a sequence 5′ to the coding region that is complementary to a portion of the vector backbone and is 15-40 nucleotides in length. In some of these embodiments, the sequence 5′ to the coding region that is complementary to a portion of the vector backbone is 20-30 nucleotides in length.

In some embodiments, the nucleotide sequence includes a sequence 3′ to the coding region that is complementary to a portion of the vector backbone and is 15-40 nucleotides in length. In some of these embodiments, the sequence 3′ to the coding region that is complementary to a portion of the vector backbone is 20-30 nucleotides in length.

In some embodiments, providing the polynucleotide includes incubating the cloning vector backbone and nucleotide sequence template in the presence of a ligase, an exonuclease, and a polymerase. In some of these embodiments, the ligase, the exonuclease, and the polymerase are present simultaneously.

In another aspect, this disclosure describes library of biological molecules produced by any embodiment of the methods summarized above.

In some embodiments, the library has a capacity of 10¹¹.

In some embodiments, the library has an efficiency of at least 20%.

In some embodiments, the library has a diversity at least 10⁸ unique members.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Distribution of unique sequences in a library produced by conventional library methods compared to distribution of unique sequences in a library produced by methods described herein. Left panel: Distribution of the number of unique peptide sequences and fraction of total peptide sequences in a commercially-available library (New England Biolabs, Inc., Ipswich, Mass., purchased from NEB (adapted from Matochko et al., Methods 2012. 58(1):47-55). Black-and-white stacked bar. The height of each segment is proportional to the fraction that each sub-population occupies in the library. For example, 5% of the library is occupied by 20 sequences, present at abundance of >30,000 copies. 150 sequences occupied nearly 20% of total reads, present at >10,000 copies, etc. Right panel: Results of the method described herein, where nearly 3.4 million unique sequences were found in a total of 3.5 million reads (on the left in purple) and fraction of those 3.4 million sequences and their occurrence in approximately 3.5 million reads sequenced. 5% of reads were occupied by approximately 170,000 unique sequences out of 3.4 million sequences, present at nearly 1 copy each and only a very few sequences were present more than once (also see table 1). This confirms that sequences in a library produced by the methods described herein are extremely uniformly distributed.

FIG. 2. Distribution of nucleotides across regions of sequence diversity. (A) Distribution of nucleotides at every position within a 39-nucleotide region of sequence diversity (SEQ ID NO:1) as previously reported (Mol Ther 2015 April; 23(4):675-682). (B) Distribution of nucleotides at every position within a 21-nucleotide region of sequence diversity (SEQ ID NO:2) in one million reads out of seven million reads using an exemplary library as described herein. Each nucleotide is distributed fairly uniformly and consistently. N=A,C,T, or G; K=T or G.

FIG. 3. Double-stranded inserts having a 20-nucleotide overlap produced a smaller number of clones compared to single-stranded inserts having a 25-nucleotide overlap.

FIG. 4. A schematic illustration of an exemplary vector used for cloning. The backbone of the illustrated vector is pUC19. The vector has a partial adenovirus genome (Ad5), a loxP site for recombination, and multiple cloning sites defined by multiple restriction enzyme cutting sites and stop codons. Cutting with Cla1 and cloning produced a seamless viral genome.

### DETAILED DESCRIPTION OF ILLUSTRATIVE EMBODIMENTS

In one aspect, this disclosure describes a method for generating a high sequence capacity library and libraries produced using the described method. The libraries exhibit quality and quantity unattainable using conventional library generating methods.

As used herein, the term “diversity” refers to the number of unique amino acid and/or nucleotide sequences that are possible within a given length of amino acid sequence and/or nucleotide sequence. For example, the diversity—i.e., the maximum number of combinations—of seven randomly ordered amino acids is 20⁷ (1.28×10⁹) and the maximum number of corresponding 21 randomly ordered nucleotides is 4²¹.

As used herein, the term “capacity” refers to the number of amino acid sequences or nucleotide sequences that the system is able to accommodate. Thus, for example, to ensure that a system can generate a library to cover all possible combinations of seven randomly ordered amino acids, the system must have a capacity of at least 1.28×10⁹ amino acid sequences or 4²¹ nucleotide sequences.

Some unique sequence can be present in multiple copies. Some sequences may be present in high copies while other sequences may be present only once. Each occurrence of a sequence is included in the term “capacity,” while duplicates of a given sequence are counted only once in the context of “diversity.” For example, culturing one clone in 10 liters of culture medium compared to culturing a billion clones in 10 liters of culture medium will produce cultures of equal capacity, but the first culture has a diversity of 1, whereas the second culture has a diversity of one billion.

As used herein, the term “efficiency” refers to the ratio of theoretically possible unique sequences produced by the library. Efficiency is a measure of the distribution of sequencing present in the library. Efficiency also can be an indicator of the scalability of the method, as explained in more detailed below.

Conventional methods for generating libraries of plasmids or recombinant viruses fail to produce the capacity of the methods described herein. Some conventional protocols use PCR-based cloning, PCR-restriction enzyme-based cloning, Gibson Assembly, and/or modified methods to generate libraries, but the diversity actually achieved is very low. Some conventional library-generating methods claim to have a diversity on the order of 10⁹ to 10¹⁰. These claims are not backed with actual analyses of diversity. Rather, the claims are based on theoretical extrapolations based on a few hundred or fewer sequences. Notably, these estimations have not been confirmed by data demonstrating that the extrapolated diversity and capacity is, in fact, attainable.

The library-generating method described herein modifies a conventional protocol to achieve previously unattainable capacity of at least 3×10⁹, such as, for example, at least 10¹⁰, at least 10¹¹, at least 10¹², or at least 10¹³, which allows one to generate a library with a diversity up to the achieved capacity. For example, creating a library of nucleotide sequences of up to 30 nucleotides, where each nucleotide has a capacity of 10¹⁰ will generate a total capacity of 3×10¹¹. Moreover, the methods described herein produce a library with reduced bias in the destribution of each candidate in the library and uniform destribution of sequence at each position among cloned sequences. Finally, the methods described herein are robust; the methods may be performed to generate libraries of nucleotides, amino acids, peptides, antibodies, proteins with site-specific mutations, aptamers, DARPins, CRISPRs, etc.

The methods described herein can generate an ultra-high-capacity library. While described herein in the context of an exemplary embodiment in which the library is plasmid-based, the methods may be performed to produce alternative libraries such as, for example, bacmid-based libraries, cosmid-based libraries, phagemid-based libraries, viral-based libraries, phage-based libraries, genomic-based libraries, and artificial-choromosome-based libraries. A plasmid-based library may be used to generate libraries of, for example, phages, recombinant viruses, or antibodies.

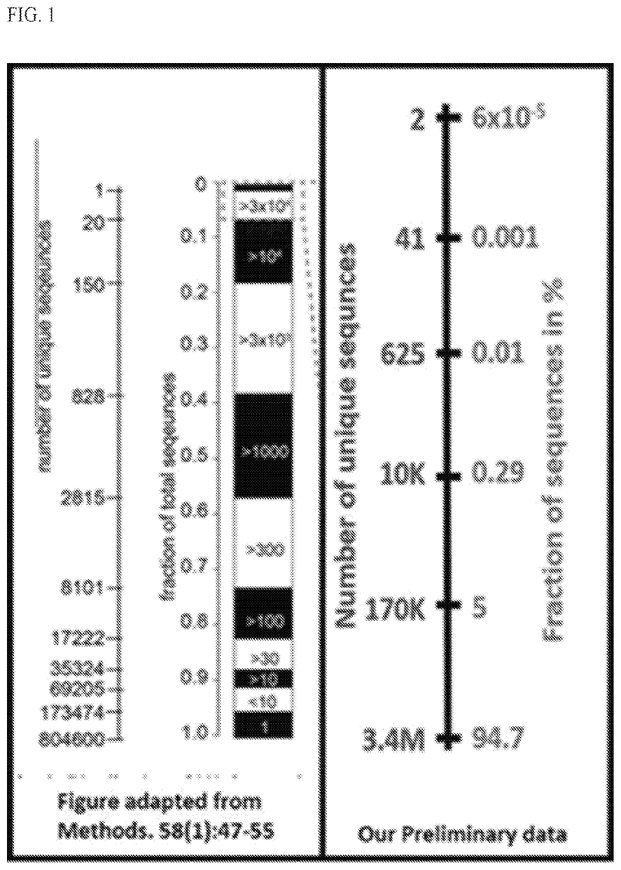

The capacity and diversity of the methods described herein have been accurately determined by high-throughput next-gen sequencing methods and analyzed by applying highly stringent criteria. The methods described herein have demonstrably produced approximately 3×10⁸ unique sequences that have been read, identifying more than 6×10⁷ unique sequences. The diversity achieved can be increased by, for example, performing more rounds of sequencing. Conventional methods (e.g., methods described in Matochko et al., Methods 2012. 58(1):47-55) produce a maximum of approximately 804,000 candidates and a ratio of unique candidates to total sequences read was less than 10% out of nearly 10 million reads sequenced. (FIG. 1, left panel).

FIG. 1 (right panel) shows the results of generating a library of plasmids—in this exemplary embodiment, a library of a shuttle vector for generating recombinant adenovirus.

More than three million reads were sequenced using Illumina-based next gen sequencing methods. More than 3.4 million of 3.5 million reads were unique sequences, producing an efficiency of greated than 98%. The efficiency of producing a diversity of 3.4 million unique sequences (>98% of reads) compares favorably against 804,000 (10% of reads) as reported previously (Matochko et al., Methods 2012. 58(1):47-55). Further, the library produced using the method described herein was sequenced to much higher depth and confirmed identification of more than 6×10⁸ candidates, which also compares favorably against 804,000 sequences. FIG. 1 therefore shows that the methods described herein produce a library with higher quality (e.g., greater diversity and/or nucleotide distribution) and higher quantity (e.g., capacity) than libraries produced using conventional methods.

The high ratio of unique sequences out of total reads sequenced (98%) indicates that the methods described herein are highly scalable. One cannot scale up conventional library-generating methods with lower ratios of unique sequences by simply scaling up the volume of reaction or by sequencing more reads. In practice, the scalability of a chemical/enzymatic reaction is not linear; rather, the scalability leads to saturation over a certain range. In order to verify and establish the extent to which the methods described herein are scalable, a second library was generated and sequenced 1/100 of the library to a depth of over 150 million paired end reads. 27,071,890 candidates with unique sequences were observed. On repeating this step with another 1/100 fraction of the same library, another 27,443,852 candidates were observed.

Next, both datasets were combined to estimate the extent to which the library-generating method was scalable and efficient. The 150 million reads of both 1/100 fractions were analsysed again. Based on the analsyis of each data set, the theroetical limit of capacity is 54,515,742 sequences (27,071,890+27,443,852) if all the sequences in both datasets were unique. However, 51,503,685 unique sequences were observed. This shows that the libraries contained 94.47% unique sequences and the overlap between libraries was only 5.5%, verifying that the library-generating methods are both efficient (98% unique sequences) and scalable (5.5% overlap). Moreover, these results suggest that the total capacity of the methods has not yet been reached.

One concern with libraries generated with methods having a large capacity is the distribution of sequences. Often, some candidates in the library are over-represented (repetitions) compared to others. For example, in one published report (Matochko et al., Methods 2012. 58(1):47-55), data reproduced in FIG. 1 demonstrates sequence bias and skewed distribution. Out of approximately 10 million reads sequenced, which consists of over 804,000 candidates, over 5 million reads (0.5 fraction of total sequences) were represented by only about 2,000 unique sequences and more than two million reads (0.2 fraction of total sequences) were represented by only about 150 candidates. The candidate with highest number representation was seen with 30,000 reads and smallest was read only once. So the range distribution (repetitions of sequences) of 804,000 candidates was 1 to 30,000.

In contrast, using a library prepared using the methods described herein, 94.6% reads out of 3.5 million were represented by approximately 94% unique sequences (FIG. 1). Candidates in our libraries ranged between 1 and 6 repetitions (FIG. 1 and Table 1), proving the distribution of candidates in libraries prepared from the methods described herein is fairly uniform. Comparing the duplications of sequences of 6 versus 30,000 shows that the methods described herein can produce library with unprecedented uniform distribution.

TABLE 1

| Candidates (sequences) | Read Counts | Total Reads |
|---|---|---|
| 2 | 6 | 12 |
| 41 | 5 | 205 |
| 625 | 4 | 2,500 |
| 10,080 | 3 | 30,240 |
| 170,601 | 2 | 341,202 |
| 3,225,061 | 1 | 3,225,061 |
| 3,406,410 | TOTAL | 3,599,220 |

Another limitation of conventional high sequence diversity libraries is biased distribution of nucleotides across the region where diversity is expected. This may happen, for example, during chemical synthesis of templates, amplification by PCR or any other methods, and/or during the cloning procedure.

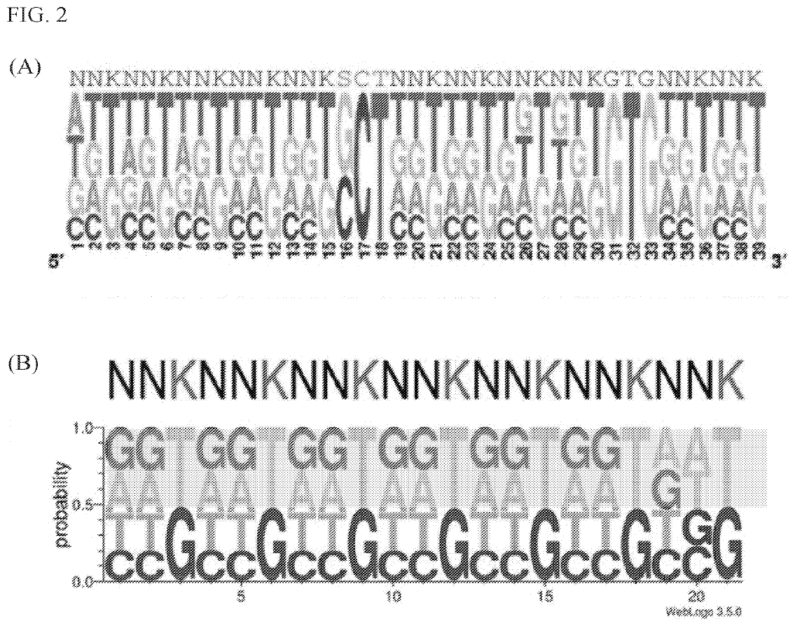

Templates were chemically synthesized (Integrated DNA Technologies, Inc., Coralville, Iowa) and cloned into a vector. Libraries were sequenced and almost one million randomly selected reads were analyzed for frequency of distribution of each nucleotide. The structure of oligo template was NNKNNKNNKNNKNNKNNKNNK (SEQ ID NO:2), where N=A,T,G or C, K=T or G and flanked by known sequences. The distribution pattern showed in FIG. 2 indicates that libraries generated using the methods described herein have consistency and uniformity, reducing bias in the sequences produced in the library (FIG. 2B). Oligonucleotide templates for cloning were chemically synthesized where each base at every ‘N’ is 25% and ‘K’ is 50% from position 1 to 21. Upon cloning the oligonucleotides, the libraries show highly consistent and uniform distribution across the 21-nucleotide region. Compared to a profile shown in FIG. 2A, where ‘N’ and ‘K’ are not seen at 25% and 50% respectively, the data in FIG. 2B are superior in quality and quantity. Chemical synthesis has an error rate of approximately 5-10% at each position. Considering this, the methods described herein effectively produce libraries with uniform distribution of sequences across the region to almost theoretical limits.

Thus, this disclosure describes a method for making a high sequence diversity library. The methods produce a system that has the capacity to produce a library of sequences with high sequence diversity. Moreover, the methods can generate a high sequence diversity library efficiently, scalably, and with minimal sequence bias. As indicated above, “efficiency” refers to the ratio of theoretically possible unique sequences produced by the library. For example, the data in FIG. 1 show that the methods described herein produced a library that included more than 97% of the possible unique sequences (right panel) whereas a library prepared using conventional methods produced a library that included approximately 10% of possible unique sequences.

The methods described herein can produce a library with efficiency of at least 20%, such as, for example, at least 30%, at least 40%, at least 50%, at least 60%, at least 70%, at least 80%, at least 90%, at least 91%, at least 92%, at least 93%, at least 94%, at least 95%, at least 96%, or at least 97%. In some applications (e.g., a library of peptides, antibodies, or specific binders), an efficiency of 20% may be sufficient. In other cases, a higher degree of efficiency may be desired. The data in FIG. 1, right panel, demonstrates that the methods described herein are capable of producing a library with a degree of efficiency that has been previously unattainable.

Library efficiency also may be an indicator of the scalability of the method. For example, a conventional library-producing method (with approximately 10% efficiency and biased sequence distribution) are not scalable. Merely increasing the volume of the culture will not appreciably increase the unique sequences detected because of the low degree of efficiency and the sequence bias since scaling up results primarily in many more copies of the high-frequency sequences. In contrast, the present methods are scalable; increasing the volume of the culture allows one to exploit the full capacity of the system. The efficiency observed and the relatively even distribution of sequences obtained using the methods described herein indicates that the additional sequences produced by scaling up the methods are more likely to be new unique sequences rather than duplicates of already-identified sequences.

The methods can employ any suitable nucleotide sequence template or insert that can be cloned into a suitable vector. Suitable vectors include any vector suitable for cloning and/or expressing polypeptides. Such vectors typically include restriction sites that allow one to insert one or more heterologous nucleotide sequences that are being cloned. A suitable vector need not have restriction enzyme cutting sites, however, if the vector includes another form of cloning site. Depending upon the application, the library generated may be a library of nucleotide sequences, a library of peptides encoded by the nucleotide sequence template, a library of antibodies, a CRISPR library, etc.

The methods described herein can generate an ultra-high-capacity library. While described herein in the context of an exemplary embodiment in which the library is plasmid-based, the methods may be performed to produce alternative libraries such as, for example, bacmid-based libraries, cosmid-based libraries, phagemid-based libraries, viral-based libraries, phage-based libraries, genomic-based libraries, and artificial-choromosome-based libraries. A plasmid-based library may be used to generate libraries of, for example, phages, recombinant viruses, or antibodies.

The nucleotide sequence template may be a single-stranded nucleotide sequence, a double-stranded nucleotide sequence, or a sequence of modified nucleotides, as may be appropriate for use in a vector appropriate for a chosen application.

The nucleotide sequence template includes sequences that overlap with sequences in the vector used for cloning. Suitable vectors include, but are not limited to, plasmids, phagemids, cosmids, artificial chromosomes, full or partial genomic DNA, phage DNA, complete or partial linear DNA, complete or partial circular DNA, etc.

In some embodiments, the nucleotide sequence template can be designed to include 21 coding nucleotide bases, plus flanking sequences that range from 15 to 40 nucleotide bases. As used herein, the “coding” nucleotide bases form a “coding region.” The “coding region” refers to that portion of the nucleotide sequence template on which the library is based. In preparing a library of peptides, the coding region encodes the peptide on which the library to be generated is based. In preparing a nucleotide sequence library, the coding region may include a nucleotide sequence that natively encodes a peptide sequence or, alternatively, may include a nucleotide sequence that does not natively encode a peptide sequence. Thus, in the context of preparing a nucleotide sequence library, it is not necessary or implied that the nucleotide sequence in the coding region natively codes a peptide sequence. At least a portion of the flanking sequences contain one or more restriction sites for cloning into vectors. In some embodiments, a flanking sequence can contain multiple restriction sites so that a single template can be suitable for cloning into multiple vectors with differing restriction sites.

In some embodiments, the flanking sequence can be 24 nucleotide bases in length. The length of the flanking sequence can, however, be modified to achieve, for example, a desired melting temperature (Tₘ.). The length of the flanking sequence also can be modified depending on the number of clones expected. For example, if only 100,000 clones with uniform distribution are expected, then one can modify the length of the flanking sequence to generate a library with a capacity closer to the expected diversity. As another example, if cloning sites of the vector or the nucleotide sequence template are A-T rich, then a longer flanking sequence may be desired to achieve efficient annealing of complementary sequences. Conversely, shorter flanking sequences may be desired if the region is G-C rich. A flanking sequence length of 24 nucleotide bases is contrary to recommendations of several cloning kit manufacturers (Takara Bio USA Inc., Mountain View, Calif.; SGI-DNA, Inc., LaJolla Calif.; New England Biolabs, Inc., Ipswich, Mass.).

The methods also can use a reaction volume that is greater than that recommended by cloning kit manufacturers. The final reaction volume can vary from about 10 μl to about 10 ml. Thus, for example, the final reaction volume can be a minimum of at least 10 μl, at least 50 μl, at least 100 μl, at least 200 μl, at least 500 μl, or at least 1 ml. The final reaction volume can be a maximum of no more than 10 ml, no more than 5 ml, no more than 2 ml, no more than 1 ml, no more than 500 μl, no more than 250 μl, no more than 200 μl, no more than 100 μl, no more than 50 μl, or no more than 20 μl. In some cases, the final reaction volume can be within a range having endpoints defined by any minimum final reaction volume listed above and any maximum final reaction volume listed above that is greater than the minimum final reaction volume.

The methods also can employ increased reaction time compared to conventional cloning methods. For example, the reaction time may be 20 minutes to 24 hours. Thus, the minimum reaction time may be at least 20 minutes, at least 30 minutes, at least 45 minutes, at least 60 minutes, at least two hours, at least three hours, at least six hours, at least 12 hours, or at least 18 hours. The maximum reaction time may be no more than 24 hours, no more than 18 hours, no more than 16 hours, no more than 12, hours, no more than eight hours, no more than six hours, no more than four hours, no more than three hours, no more than two hours, no more than 60 minutes, no more than 40 minutes, or no more than 30 minutes. In some embodiments, the reaction time may fall within a range having endpoints defined by any minimum reaction time listed above and any maximum reaction time listed above that is greater than the minimum reaction time. For example, in some embodiments, the reaction time may be from 30 minutes to four hours, such as, for example, from 45 minutes to two hours.

The methods also can include supplementing the cloning reaction with additional enzymes and buffers that are not recommended in conventional cloning methods. For example, the reaction mixture can include an exonuclease that digests in the 5′-to-3′ direction. One exemplary suitable exonuclease is T5 exonuclease. As another example, the reaction mixture can include a ligase that is capable of ligating annealed fragments that are within suitable proximity with one another and with the ligase. Exemplary suitable ligases include, but are not limited to, 9° N™ DNA Ligase, taq ligase, Tth ligase, T4-DNA ligase. As yet another example, the reaction mixture can include a polymerase suitable for use under the desired reaction conditions. Exemplary suitable polymerases include, but are not limited to, Bst DNA polymerase, large fragment of Bst DNA polymerase, taq DNA polymerase, similar polymerases used for PCR or isothermal amplification. In some embodiments, the reaction mixture includes an exonuclease and a ligase and a polymerase.

The reaction conditions can be selected based on the composition of the reaction mixture. For example, the reaction conditions can include an incubation at a temperature of from 12° C. to 72° C., but may include one or more intervening incubations outside of this range. In general, however, the minimum incubation temperature can be at least 12° C., at least 20° C., at least 27° C., at least 30° C., at least 37° C., or at least 50° C. The maximum incubation temperature can be no more than 72° C., no more than 65° C., no more than 60° C., no more than 55° C., or no more than 50° C. The incubation temperature also can fall within a range having endpoints defined by any minimum incubation temperature recited above and any maximum incubation temperature recited that is greater than the minimum incubation temperature. Thus, the reaction may be performed at a temperature of from 12° C. to 72° C., such as, for example, from 37° C. to 60° C., or at 50° C. In one particular embodiment, the reaction conditions can include incubating the reaction mixture for 15 minutes to two hours at a temperature of, for example, 50° C.

In many instances, the precise temperature of the incubation can depend, at least in part, on the specific enzymes chosen for the reaction. Also, the incubation may be performed at more than a single temperature. For example, a reaction using T4 ligase, T5 exonuclease, and Taq DNA polymerase may employ incubations at 37° C., 65° C., and 16° C. to complete one reaction.

The reaction conditions may include one or more intervening incubations at other temperatures for certain durations of time. Such an intervening incubation may be designed to melt annealed polynucleotides and may, therefore, occur outside the ranges listed above. An intervening incubation may be performed at a temperature of at least 72° C., such as, for example, at least 80° C., at least 85° C., at least 90° C., or at least 95° C. An intervening incubation may be performed at a temperature of no more than 100° C., such as, for example, no more than 95° C., no more than 90° C., no more than 85° C., no more than 80° C., no more than 75° C., or no more than 72° C.

An intervening incubation may be performed for from five minutes to 30 minutes, but may be performed for periods longer than 30 minutes, although intervening incubation times greater than 30 minutes may not appreciably improve the yield of the reaction. Thus, a minimum incubation time for an intervening incubation may be at least five minutes, at least ten minutes, at least 15 minutes, or at least 20 minutes. In some embodiments, the maximum incubation time for an intervening incubation may be no more than 30 minutes, no more than 25 minutes, no more than 20 minutes, no more than 15 minutes, or no more than 10 minutes. The incubation time of an intervening incubation may fall within a range having endpoints defined by any minimum intervening incubation time listed above and any maximum intervening incubation time listed above that is greater than the minimum intervening incubation time.

An intervening incubation may be followed by incubation on ice in order to promote re-annealing of melted polynucleotide strands.

Thus, for example, an intervening incubation may include an incubation at 72° C. for five minutes, followed by cooling on ice for five minutes.

After the cloning reaction, the cloning products are isolated to remove enzymes and salts from the buffer. The cloning products can be transformed into a suitable host cell for amplification. The suitable host call may be a bacterium, a yeast, or other host cell appropriate for the selected vector type. For example, a bacterium may be used as a suitable host cell for, for example, a phage-based or plasmid-based vector. A yeast cell (e.g., Saccharomyces cerevisiae) may be a suitable host cell for a plasmid-based or artificial-chromosome-based vector. Mammalian cells may be suitable host cells for generating libraries by direct expression of peptides and/or assembling mammalian recombinant viruses.

Transformation may be performed using conventional methods for the selected host cell. For example, electroporation may be used to transform bacterial cells. When the host cell is a bacterium, the transformed cells are cultured for a suitable time under conventional conditions for the host cell and transforming vector. After a suitable incubation, the library (e.g., plasmid-based or phage-based) can be extracted using conventional methods.

In some embodiments, the cloning products may be amplified in vitro using conventional in vitro methods for amplifying polynucleotides.

Members of the libraries may be sequences using any conventional sequencing method, then analyzed to determine the distribution of sequences in the library.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

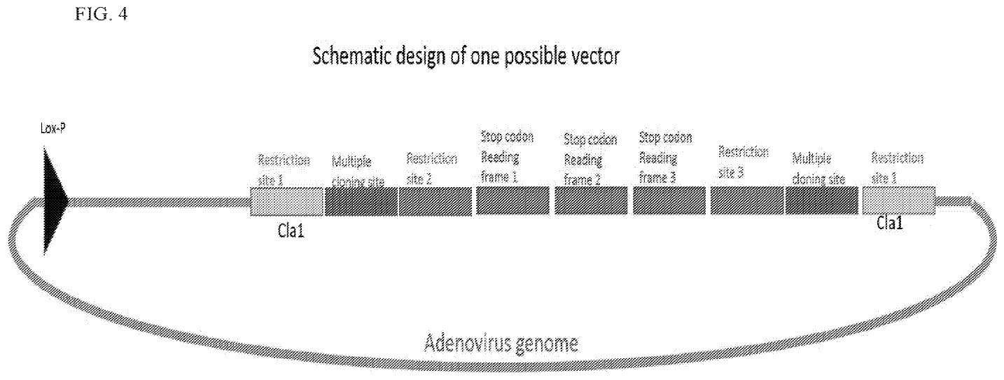

A plasmid based adenoviral shuttle vector was designed and constructed (FIG. 4). This shuttle vector has a multiple cloning sites. The vector was digested with Cla1 restriction enzyme to linearize the vector. The sequences flanking the Cla1 sites are derived from adenovirus type 5 virus. The final cloned product will produce a seamless adenovirus genome sequence.

Oligonucleotide inserts for cloning were chemically synthesized. The oligos (82 nucleotides in length) had following features: a) they have a part of adenovirus genome flanking the AB-loop region (AB-loop region is 21 nucleotide bases in length). The region corresponding to AB-loop region contains sequence diversity in the format NNK, where N=A,T,G, or C and K=T or G. Overlapping sequences were 24 nucleotide bases on either end (underlined). The final sequence is in the format:

(SEQ ID NO: 3)
aactttgtggaccacaccagctccatctcctaacNNkNNkNNkNNkNNk
NNkNNkgatgctaaactcactttggtcttaaca.

A oligonucleotide complementary to aactcactttggtcttaaca (nucleotides 1-10 of SEQ ID NO:3) was synthesized. 10 nM of the 82-nt oligo and the 20-nt oligo were annealed by mixing them in equimolar ratio, heating to 95° C. for five minutes and slowly cooled to room temperature. Annealed oligonucleotides, which produce a partial double strand, were filled in with E. coli DNA polymerase (New England Biolabs, Inc., Ipswich, Mass.).

The linearized vector and insert were mixed in a 1:2 molar ratio and incubated with NEBUILDER enzyme (New England Biolabs, Inc., Ipswich, Mass.) in a 20 μl reaction for 30 minutes at 50° C., heated to 72° C. for five minutes, then cooled down on ice for five minutes. T5 exonuclease was added and the mixture was incubated for 15 minutes at 50° C. Bst DNA polymerase large fragment (New England Biolabs, Inc., Ipswich, Mass.) and Taq DNA ligase (New England Biolabs, Inc., Ipswich, Mass.) were added and incubated at 50° C. for another 30 minutes.

In parallel, additional vectors (cloned vectors) were produced using the same methods, but based on oligonucleotides with different lengths of overlapping sequences: 20 overlapping nucleotide bases (nucleotides 1-20 of SEQ ID NO:3) and 25 overlapping nucleotide bases (nucleotides 1-25 of SEQ ID NO:3), keeping all other features the same. These vectors (cloned plasmids) were produced in the same manner as the first vector.

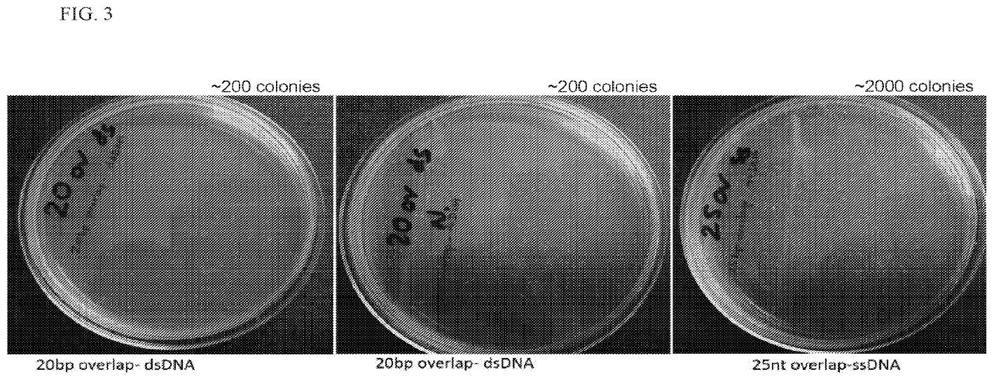

Each vector was purified, then separately electroporated into electrocompetent cells (Thermo Fisher Scientific, Waltham, Mass.) according to the manufacturer's instructions, then cultured to get sufficient bacterial growth. After one hour, 100 μl of the electroporated bacteria were plated onto LB-Agar plates containing ampicillin (100 μg/ml). Plates were cultured overnight and pictures were taken. Results are shown in FIG. 3.

Plasmids were extracted (Qiagen hi-speed maxi-prep kit; Qiagen, Hidden, Germany) and sequenced using the Illumina platform. 3.5 million reads were produced in the first run using Illumina miSeq platform. Later, 150 million reads were sequenced on Illumina Hi-seq platform.

Paired-end sequencing data were analyzed by trimming the sequences flanking the 21-nucleotide AB-loop region. The resulting sequences were sorted in decreasing order of the number they were sequenced. The modules used in this analysis were “PEAR, Cutadapt and Fastx collapser”. These modules were available in the University of Minnesota supercomputing facility.

Distribution of nucleotides at each position was analyzed and Sequence logos (figure of distribution of nucleotides at each position) were created by weblogo (http://weblogo.berkeley.edu/). One million reads out of 3.5 million sequenced reads were randomly chosen to perform this analysis.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for producing a library of biological molecules, the method comprising:
providing a polynucleotide comprising:
a cloning vector backbone;
a recombination site;
one or more cloning sites;
a nucleotide sequence template, the nucleotide sequence template comprising a coding region, the coding region comprising either the biological molecule on which the library is based or encoding the biological molecule on which the library is based;
a sequence 5′ to the coding region that is complementary to a portion of the vector backbone; and
a sequence 3′ to the coding region that is complementary to a portion of the vector backbone;
introducing the polynucleotide into a host cell, thereby producing a genetically modified cell;
incubating the genetically modified cell under conditions to allow the polynucleotide to replicate;
collecting genetically modified cells;
isolating vector from the collected cells; and
sequencing the coding region.

2. A method for producing a library of biological molecules, the method comprising:
providing a polynucleotide comprising:
a cloning vector backbone;
a recombination site;
one or more cloning sites;
a nucleotide sequence template, the nucleotide sequence template comprising a coding region, the coding region comprising either the biological molecule on which the library is based or encoding the biological molecule on which the library is based;
a sequence 5′ to the coding region that is complementary to a portion of the vector backbone; and
a sequence 3′ to the coding region that is complementary to a portion of the vector backbone;
amplifying the polynucleotide in vitro;
collecting the amplified polynucleotides; and
sequencing the coding region of the amplified nucleotides.

3. The method of claim 1, wherein the sequence 5′ to the coding region that is complementary to a portion of the vector backbone is from 15-40 nucleotides in length.

4. The method of claim 3, wherein the sequence 5′ to the coding region that is complementary to a portion of the vector backbone is 20-30 nucleotides in length.

5. The method of claim 1, wherein the sequence 3′ to the coding region that is complementary to a portion of the vector backbone is from 15-40 nucleotides in length.

6. The method of claim 5, wherein the sequence 3′ to the coding region that is complementary to a portion of the vector backbone is 20-30 nucleotides in length.

7. The method of claim 1, wherein at least one of the sequence 5′ to the coding region and the sequence 3′ to the coding region comprises:
a single-stranded nucleotide sequence;
a double-stranded nucleotide sequence; or
a modified nucleotide.

8. The method of claim 1, wherein providing the polynucleotide comprises:
incubating the cloning vector backbone and nucleotide sequence template in the presence of a ligase, an exonuclease, and a polymerase.

9. The method of claim 8, wherein the ligase, the exonuclease, and the polymerase are present simultaneously.

10. The method of claim 1, wherein sequencing the coding region comprises:
translating the nucleotide sequence of the coding region; and
sequencing the translated peptide.

11. A library of biological molecules produced by the method of claim 1.

12. The library of biological molecules of claim 11, wherein the library has a capacity of at least 10¹¹.

13. The library of biological molecules of claim 11, wherein the library has a capacity of up to 10¹³.

14. The library of biological molecules of claim 11, wherein the library has an efficiency of at least 20%.

15. The library of biological molecules of claim 11, wherein the library has:
a capacity of at least 10⁸; and
a diversity at least 10⁸ unique members.

16. The method of claim 2, wherein the sequence 5′ to the coding region that is complementary to a portion of the vector backbone is from 15-40 nucleotides in length.

17. The method of claim 16, wherein the sequence 5′ to the coding region that is complementary to a portion of the vector backbone is 20-30 nucleotides in length.

18. The method of claim 2, wherein the sequence 3′ to the coding region that is complementary to a portion of the vector backbone is from 15-40 nucleotides in length.

19. The method of claim 18, wherein the sequence 3′ to the coding region that is complementary to a portion of the vector backbone is 20-30 nucleotides in length.

20. The method of claim 2, wherein at least one of the sequence 5′ to the coding region and the sequence 3′ to the coding region comprises:
a single-stranded nucleotide sequence;
a double-stranded nucleotide sequence; or
a modified nucleotide.

21. The method of claim 2, wherein providing the polynucleotide comprises:
incubating the cloning vector backbone and nucleotide sequence template in the presence of a ligase, an exonuclease, and a polymerase.

22. The method of claim 21, wherein the ligase, the exonuclease, and the polymerase are present simultaneously.

23. The method of claim 2, wherein sequencing the coding region comprises:
translating the nucleotide sequence of the coding region; and
sequencing the translated peptide.

24. A library of biological molecules produced by the method of claim 2.

25. The library of biological molecules of claim 24, wherein the library has a capacity of at least 10¹¹.

26. The library of biological molecules of claim 24, wherein the library has a capacity of up to 10¹³.

27. The library of biological molecules of claim 24, wherein the library has an efficiency of at least 20%.

28. The library of biological molecules of claim 24, wherein the library has:
a capacity of at least 10⁸; and
a diversity at least 10⁸ unique members.
